(19) United States
(12) Patent Application Publication
(10) Pub. No.: US 2021/0313063 A1
(43) Pub. Date: Oct. 7, 2021

(54) MACHINE LEARNING MODELS FOR GAPS IN CARE AND MEDICATION ACTIONS

(71) Applicant: Clover Health, Jersey City, NJ (US)

(72) Inventors: Kevin Holub, San Francisco, CA (US); Christopher James Lauinger, Golden, CO (US); Melanie Goetz, Oakland, CA (US)

(21) Appl. No.: 16/842,624

(22) Filed: Apr. 7, 2020

(51) Int. Cl. G16H 50/20; G16H 15/00; G06N 20/00; G06N 5/04; G06K 9/62

(52) U.S. Cl. CPC G16H 50/20; G16H 15/00; G06K 9/6263; G06N 5/04; G06K 9/6221; G06N 20/00

(57) ABSTRACT

The present disclosure describes methods and systems for machine learning models utilized for identifying issues with medications and gaps in care. The present disclosure also describes methods and systems for machine learning models utilized to provide recommended actions for addressing the issues with medication and gaps in care. These methods and systems utilize machine learning models that are trained to identify issues with medications and gaps in care, as well as provide recommended actions for those medications issues and gaps in care. The models are trained with data from disparate sources that are aggregated and formatted to be utilized in these models.

### BACKGROUND

Identifying gaps in care and providing recommended actions for medications for a medical patient may be desired. Described herein are improvements in technology and solutions to problems that may be used, among other things, to assist in identifying gaps in care and determining medication actions.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates a schematic diagram of an example environment for the utilization and implementation of surfacing recommended actions for medication and identifying gaps in care.

FIG. 2 illustrates a conceptual diagram of example components of identifying gaps in care architecture and systems.

FIG. 3 illustrates a conceptual diagram of example components of systems for surfacing recommended actions for medication.

FIG. 4 illustrates a flow diagram of an example process for identifying gaps in care.

FIG. 5 illustrates a flow diagram of an example process for surfacing recommended actions for medication.

FIG. 6 illustrates an example user interface utilized for confirming or rejecting a gap in care recommendation determined utilizing the machine learning models described herein.

FIG. 7 illustrates an example user interface for confirming or rejecting a recommended action for medication determined utilizing the machine learning models described herein.

FIG. 8 illustrates a conceptual diagram of input data, components of machine learning models, and output data associated with identifying gaps in care and surfacing recommended actions for medication.

FIG. 9 illustrates a flow diagram of an example process for utilizing machine leaning models for identifying gaps in care.

FIG. 10 illustrates a flow diagram of an example process for utilizing machine learning models for surfacing recommended actions for medication.

### DETAILED DESCRIPTION

Systems and methods for utilizing machine learning models for identifying gaps in care and surfacing recommended actions for medication are described herein. Accurately identifying gaps in care and surfacing recommended actions for medication relies on data to detect patterns and identify anomalies that would otherwise be unapparent to a medical service provider. This process may be optimized and improved through the use of large, complex data and machine learning to recognize patterns, identify anomalies, and provide accurate probabilities of action based on a recommended action. For example, data from disparate sources that may not traditionally be viewed for identifying gaps in care data (e.g. laboratory data, medical record data, and geographical data) may be combined into larger data sets. This data may then be utilized by machine learning models and data analytic pipelines to identify gaps in care that may otherwise go unnoticed. Similar approaches may be used to identify issues with medication and surface a recommended action to solve those issues. These identified gap(s) in care and recommended action(s) for medication may be confirmed or rejected by a medical service provider (e.g. medical service provider, nurse practitioner and/or medical service provider's assistant). In examples, these identified gap(s) in care and recommended action(s) for medication may be surfaced and presented to the medical service provider, wherein the gap(s) in care and/or the recommended action(s) for medication may be confirmed or rejected. In these and other examples, the confirmation or rejection of the gap(s) in care and/or recommended action(s) for medication may be used to update the data and machine learning models to improve accuracy for future surfacing of gaps in care identification and/or medication recommended action(s).

The present innovation is directed to systems and methods that generate machine learning models configured to identify gap(s) in care and/or determine recommended action(s) for medication in a medical patient, utilize the trained machine learning models to determine a likelihood that the gap(s) in care may be successfully addressed and/or the recommended action(s) for medication may be taken for the medical patient, and surface the gap(s) in care or recommended action(s) to a medical service provider. By way of example, machine learning models may be trained on a large data set, wherein individual machine learning models may be trained to determine gap(s) in care and/or recommended action(s) for medication for a medical patient.

The data used to train the machine learning models may be received at a database and/or data pipeline from multiple disparate data sources via a computing network. On their own, the data from the disparate sources are likely to exist in disparate formats. However, the data may be formatted into coherent data structures and formats. These data structures may be multi-dimensional and may include associated meta-data that may be unpacked and formatted within the data structures. The disparate data sources may include annotations. The data structures may be formatted into one or more model features that are configured to be input into machine learning models. Formatting data into model features may include several data manipulations. For example, data may be standardized and/or normalized to bring data with different scales into similar and/or the same scale(s). New data may be generated in appropriate scales using other techniques appropriate for machine learning models.

Data standardization is the process of rescaling features to have the properties of a Gaussian distribution where the mean is equal to zero and the standard deviation is equal to one. Data normalization is the process of rescaling features such that the range of data is fixed. For example, the range may be fixed between zero and one, and normalized based on a sigmoid function. In other examples, the range may be fixed between zero and ten, and normalized based on a rectified linear unit function. While these standardization and normalization approaches are discussed, other approaches may be utilized.

The formatted data and/or the model features may be input into machine learning models. The machine learning models may be individually trained to identify gaps in care and/or determine medication action(s). Upon receiving the input, the machine learning models may format the input to produce formatted, output data. The output data may include, for example, a probability and/or confidence value for recommended action(s), and/or that a medical patient is associated with gaps in care.

Additionally, or alternatively, the output data, including the probability and confidence interval data, may be sent by a first computing device executing a first application and received by a second computing device executing a second application. The computing device executing an application that receives the output data may also receive an indication that the medical patient that the output data is associated with will be seen by a medical service provider at a given time. The output data may then be surfaced and displayed as a notification, and may include the identified gap(s) in care and recommended action(s) for medications.

The medical service provider may receive the notification of the potential gap(s) in care and/or recommended action(s) for medications and may decide to confirm or reject the identified gap(s) in care and/or recommended action(s) for medications. This process may occur during a patient encounter, such as a doctor's clinic visit. In other examples, the encounter may occur independent of an in-person medical service provider and patient interaction, such as telehealth consulting and/or asynchronous medicine, or other forms of medical practice. The confirmation or rejection of the gap(s) in care and recommended action(s) for medications may lead to updating the data in the patient file as well as the database and provides feedback on the models that generate the identified gap(s) in care and/or recommended action(s) for medications. If another gap in care and recommended action for medication has been surfaced, it may be displayed after the first gap(s) in care and/or recommended action(s) for medication, after which the process may repeat until there are no more gap(s) in care and/or recommended action(s) for medication left to confirm or reject.

The machine learning models may be configured to identify gap(s) in care and recommended action(s) for medication, wherein the individual ones of the machine learning models are trained to receive model features and output data indicating a probability that at least one of the gap(s) in care are identified, and/or the one or more action(s) for medication in a medical patient should occur. For example, colorectal cancer screening via colonoscopy recommendations follow age determined guidelines. These guidelines may include recommendations such as colonoscopies that may be performed every ten years, a computed tomography (CT) colonography (virtual colonoscopy) that may be performed every five years, and/or a flexible sigmoidoscopy that may be performed every five years. In examples, the recommendations may indicate that screening should begin at 45 years of age for those individuals with an average risk of colorectal cancer, but may be performed prior to 45 years of age for those at an increased risk of colorectal cancer. Examples of individuals who are at an increased risk of colorectal cancer may include those individuals with a family history of colorectal cancer of colon polyps, a personal history of colorectal cancer of polyps, a personal history of inflammatory bowel disease, a known family history of a hereditary colorectal cancer syndrome such as familial adenomatous polyposis or lynch syndrome, and/or a personal history of radiation to the abdomen or pelvic area to treat a prior cancer. In examples, the machine learning models may probe data related to a medical patient's age and medical history, and cross-reference that data against data on medical procedures to determine if the medical patient should undergo colorectal cancer screening. Additionally, and/or alternatively, the machine learning models may determine whether there is a gap in care in receiving a colonoscopy within a set time frame based on colorectal cancer screening guidelines. If there is indeed a gap in care for the medical patient, the machine learning models may surface this as an identified gap in care and may produce a recommendation for scheduling a colonoscopy for the medical patient. In other examples, a recommendation for actions(s) for medication may include identifying patient(s) who require a change in current medication dosage. For example, the machine learning model may recommend changing a medication's refill quantity. In examples, a medical patient may interact with a medical service provider to discuss blood pressure management where the machine learning models may identify that the patient, who is currently on 500 mg of metformin, has incurred an increase in fasting glucose and HbA1c levels over the past 18 months. In response to this information, the machine learning models may surface a recommendation to increase the patient's dosage of metformin to 500 mg twice per day.

The models for identifying gaps in care may have overlapping and/or independent features with models for surfacing recommended action(s) for medications. The selected features, normalization, standardization, and/or transformation of data may be similar and/or orthogonal across different gaps in care and recommended action(s) for medication focused models.

In examples, the data that is used to train and execute the machine learning models may include, but is not limited to, data from medical records, chart codes, Centers for Medicare & Medicaid Services data, International Codes for Diagnosis data, medication data, and/or laboratory data. Additionally, or alternatively, data may be prioritized and/or weighted for individual models. Data prioritization and/or weighting may be performed based upon predefined criteria, wherein the predefined criteria includes, but is not limited to, at least one of documented International Classification of Disease code(s), medication for singular disease, and/or laboratory value(s) that define a medical diagnosis. The prioritization and/or weighting may also be determined through the processes of training and/or testing the machine learning models. For example, the prioritization and/or weighting may occur by determining the impact of a data type on the output data, determining that the impact satisfies a threshold impact, and prioritizing the data type concurrent with the effect that data has on the outcome.

The identification of gap(s) in care or action(s) associated with medications may include determining missed appointments, intervals of time without procedures, patient medical history such as conditions, medications, and/or lab results, and determining a probability for successful implementation of a recommendation associated with the gap(s) in care and/or the action(s) for medication in the medical patients. In these examples, wherein sending the notification comprises sending the notification based at least in part on the probability satisfying a threshold probability for successfully addressing the at least one of the haps in medical care or the one or more actions.

The medical service provider may confirm and/or reject the identified gap(s) in care and/or recommended action(s) for medication. This decision may be added to the medical patient's record and may be coded as a true-positive, false-positive, true-negative, or false-negative. In examples, the decision may be fed back to the database. The machine learning models may be retrained, updated, and/or honed-in over time by receiving feedback. In examples, the machine learning models may receive feedback data over a period of time and use this data to retrain features of the machine learning models.

Additional details are described below with reference to several example embodiments.

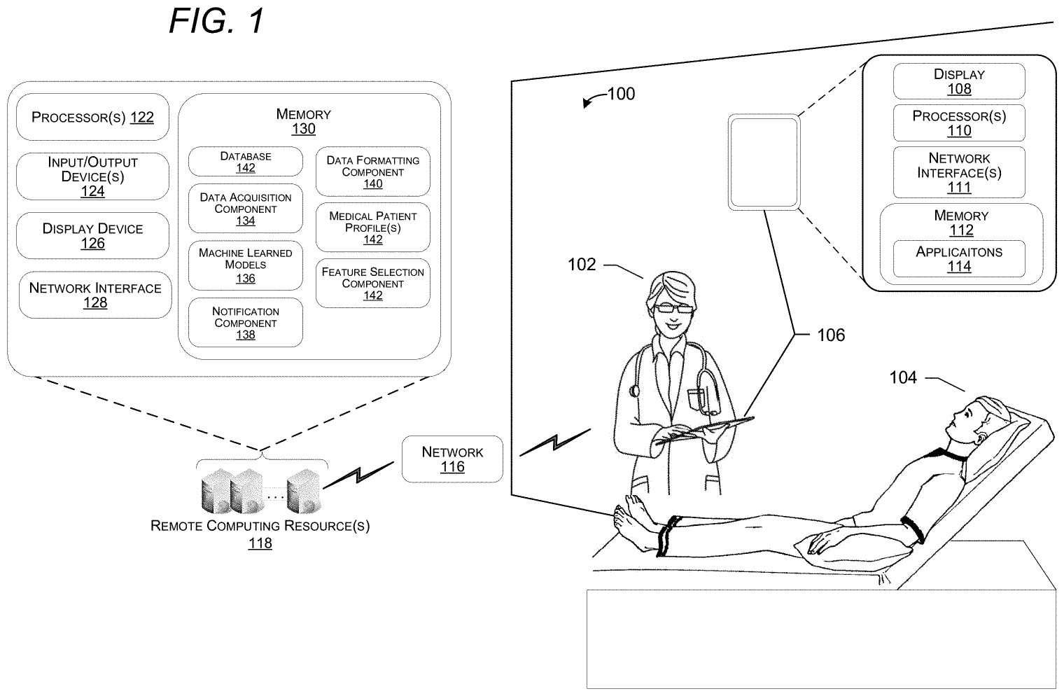

FIG. 1 illustrates a schematic of an example environment 100 for the utilization and implementation of surfacing recommended actions for medication and identifying gaps in care architecture and systems. The environment may include a medical service provider 102 (e.g. medical service provider, nurse practitioner and/or medical service provider's assistant), a medical patient 104, and a computing device 106. The computing device 106 may include a display 108, one or more processor(s) 110, one or more network interface(s) 111, and memory 112. The memory 112 may include one or more components such as one or more application(s) 114. The computing device 106 may connect to one or more remote computing resource(s) 118 through a network 116. The remote computing resource(s) may include one or more components, such as one or more processors 122, input and/or output device(s) 124, a display device 126, a network interface 128, and memory 130. The memory 130 may contain a database 132, a data acquisition component 134, machine learned models 136, a notification component 138, a data formatting component 140, medical patient profiles 142, and/or a feature selection component 144.

The remote computing resource 118 may house the hardware and software that may generate machine learning models 136 configured to identify gaps in care and surface recommended action(s) for medication. In examples, the remote computing resource 118 may utilize the trained machine learning models 136 to determine a likelihood that action will be taken based on the identified gap(s) in care and/or surfaced recommended action(s) for medication in a medical patient 104, and surface those identified gaps in care and/or recommended actions for medication to the medical service provider 102 on a computing device 106 that may be running the application 114 housed in the memory 112 and executed by the processors 122. This application 114 may be, at least in part, designed to receive the notification of identified gap(s) in care and/or recommended action(s) for medication sent by the notification component 138 and show the identified gap(s) in care and/or recommended action(s) for medication to the medical service provider 102 through the display 108. The machine learning models 136 may be trained on a large data sets stored in the database 132, wherein individual machine learning models may be trained to determine a likelihood that that action will be taken based on the identified gap(s) in care and/or surfaced recommended action(s) for medication in a medical patient 104.

The data used to train the machine learning models may be received at a database 132 from multiple disparate data sources via a computing network 116. On their own, the data may exist in disparate formats. However, the data may undergo formatting to create coherent data structures and formats. These formats may be multi-dimensional and may include meta-data that may be unpacked and formatted. In examples, the disparate data sources may include annotations. The data structures maybe formatted into model features that may be configured as inputs to the machine learning models through a feature selection component 144. Formatting data into model features may include several data manipulations. For example, data may be standardized and/or normalized to bring data with different scales into similar and/or the same scale(s).

Additionally, or alternatively, the formatted data may be input into the machine learned models 136, wherein the identification of gap(s) in care and/or surfacing of recommended action(s) for medication may occur. The identification of gap(s) in care and/or surfacing of recommended action(s) for medication output data, which may include a probability and/or confidence value, may be sent to the notification component 138. The notification component may send the information through the network 116 to the remote device, which then may surface the identified gaps in care and/or surfaced recommended action(s) for medication through the application 114. In examples, the notification component may display the recommended action(s) to the medical service provider 102. The medical service provider may confirm or reject the identified gap(s) in care and/or surfaced recommended action(s) for medication. The decision data may be sent through the network 116 to the remote computing resource(s) 118, routed through the data acquisition component 134, and/or stored in the database 132 and/or medical patient profiles 142.

As used herein, a processor, such as processor(s) 110 and/or 122, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 110 and/or 122 may include a graphics processing unit (GPU), a microprocessor, a digital signal processor or other processing units or components known in the art. Alternatively, or additionally, the functionally described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 110 and/or 122 may possess its own local memory, which also may store program components, program data, and/or one or more operating systems.

The memory 112 and/or 130 may include volatile and nonvolatile memory, removable and non-removable media implemented in any method or technology for storage of information, such as computer-readable instructions, data structures, program component, or other data. Such memory 112 and/or 130 includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 112 and/or 130 may be implemented as computer-readable storage media (“CRSM”), which may be any available physical media accessible by the processor(s) 110 and/or 122 to execute instructions stored on the memory 112 and/or 130. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information, and which can be accessed by the processor(s) 122.

Further, functional components may be stored in the respective memories, or the same functionality may alternatively be implemented in hardware, firmware, application specific integrated circuits, field programmable gate arrays, and/or as a system on a chip (SoC). Further, while not illustrated, each respective memory, such as memory 112 and/or 130, discussed herein may include at least one operating system (OS) component that is configured to manage hardware resource devices such as the network interface(s), the I/O devices of the respective apparatuses, and so forth, and provide various services to applications or components executing on the processors. Such OS components may implement a variant of the FreeBSD operating systems as promulgated by the FreeBSD Project; other UNIX or UNIX-like variants; a variation of the Linux operating system as promulgated by Linus Torvalds; the FireOS operating system from Amazon.com Inc. of Seattle, Wash., USA; the Windows operating system from Microsoft Corporation of Redmond, Wash., USA; LynxOS as promulgated by Lynx Software Technologies, Inc. of San Jose, Calif.; Operating System Embedded (Enea OSE) as promulgated by ENEA AB of Sweden; and so forth.

The network 116 may enable communications between the components and/or devices shown in environment 100 and/or with one or more other remote systems and other networked device(s). Such network(s) 116 may include one or more network interface controllers (NICs) or other types of transceiver devices to send and receive communications over the network 108.

For instance, each of the network(s) 116 may include a personal area network (PAN) component to enable communications over one or more short-range wireless communication channels. For instance, the PAN component may enable communications compliant with at least one of the following standards IEEE 802.15.4 (ZigBee), IEEE 802.15.1 (Bluetooth), IEEE 802.11 (WiFi), or any other PAN communication protocol. Furthermore, each of the network(s) 116 may include a wide area network (WAN) component to enable communication over a wide area network.

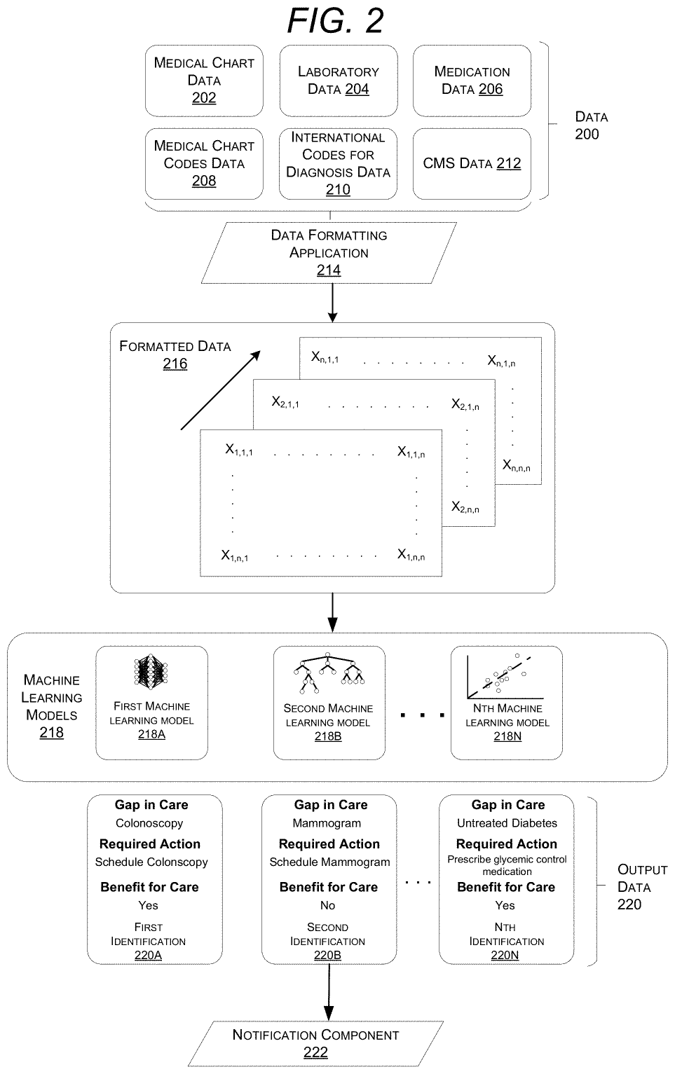

FIG. 2 illustrates a conceptual diagram of example components of identifying gaps in care architecture and systems. Data 200 from disparate sources may include at least one of medical chart data 202, laboratory data 204, medication data 206, medical chart codes 208, international codes for diagnosis data 210, and/or Centers for Medicare and Medicaid Services (CMS) data 212. The data may be fed into a data formatting application 214, where the data may be formatted into model features configured to be input into the machine learning models. The formatted data 216 may be multidimensional data that occupies N dimensional space and may be formatted to fit individual machine learning models. The formatted data may be fed into machine learning models 218A-218N (hereinafter referred to individually as “item 218A,” “item 218B,” etc., and generically as “item 218”), wherein the individual machine learning models may be trained to identify gaps in care. The machine learning models generate output data 220A-220N (herein after referred to individually as “item 220A”, “item 220B”, etc., and generically as “item 220). The output data 220 may include data regarding gaps in care (e.g. missing a diagnostic screening test and/or medication for a diagnosed illness), and/or additional data, such as probability and/or threshold data. The output data 220 may be sent to the notification component 222 which may transfer the notification from the remote computing resource 118 to the computing device 106 via the network 116 to display the notification to the medical service provider 102.

The medical chart data 202 may include at least one of, but not limited to, data regarding family health history, diet data, exercise, sexual history, smoking, alcohol consumption, immunization records, motor development, cognitive, intellectual, age, sex, race, religion, occupation, surgical history, obstetric history, allergies, hospital admissions, specialist consultations, and/or physical checkup visits. Laboratory data 204 may include at least one of, but not limited to, data regarding cell counts (e.g., red blood cell count, hematocrit, neutrophil count, lymphocyte count, monocyte count, eosinophil count, basophil count), cardiac markers (e.g., troponin I, troponin T, myoglobin), general chemistries (e.g., albumin, alkaline phosphatase, anion gap, ammonia, ALT, AST, bilirubin, blood urea nitrogen, creatinine, creatine, calcium, chloride, carbon dioxide, ethanol, folic acid, glucose, HDL, LDL, iron, iron binding capacity, lactic acid, lactate, lipase, magnesium, osmolarity, parathyroid hormone, phosphorous, sodium, T3, T4, TSH, uric acid, triglycerides), urine data (e.g., color, specific gravity, pH, sodium, potassium, protein, amylase, creatinine, magnesium), and/or coagulation data (e.g., thrombin time, platelets, plasminogen, fibrinogen, bleeding time. Medication data 206 may include at least one of, but not limited to, data regarding medication name, medication class, dosage, refill times, route of administration, drug interactions, and/or contraindications. Medical chart codes data 208 may include at least one of, but not limited to, data regarding chart codes for tests, surgeries, evaluations, procedures, surgeries, and/or prescriptions. International codes for diagnosis (ICD) data 210 may include at least one of, but not limited to, data regarding codes for diagnoses, symptoms, and procedures through the ICD coding system.

The data formatting application 214 may receive the data 200 that may be derived from multiple disparate data sources via the computing network 116. On its own, the data from the disparate sources is likely to exist in disparate formats. However, the data may be formatted into coherent data structures and formats in the data formatting application 214. The data structures may be multi-dimensional and may include associated meta-data that may be unpacked and formatted within the data structures. The data structures may be formatted into model features that may be configured to be inputs to machine learning models. Formatting data into model features may require several data manipulations. For example, data may be standardized and/or normalized to bring data with different scales into similar and/or the same scale(s). Data standardization is the process of rescaling features to have the properties of a Gaussian distribution where the mean is equal to zero and the standard deviation is equal to one. Data normalization is the process of rescaling the features such that the range of the data is fixed. For example, the range may be fixed between zero and one and normalized based on a sigmoid function. In other examples, the range may be fixed between zero and ten, and normalized based on a rectified linear unit function. While these standardization and normalization approaches are discussed, other approaches may be utilized.

Data standardization is the process of rescaling the features, such that they will have the properties of a Gaussian distribution where the mean is equal to zero and the standard deviation is equal to one. Data normalization is the process of rescaling the features such that the range of the data is fixed. For example, the range can be fixed between zero and one, and normalized based on a sigmoid function. In other examples, the range can be fixed between zero and 10, and normalized based on a rectified linear unit function. While these standardization and normalization approaches are discussed, many others are contemplated. New data may be generated appropriate scales using other techniques appropriate for machine learning models.

The data formatting application 214 may output formatted data 216. The formatted data 216 may be multi-dimensional and may exist in N dimensional space. The formatted data 216 may exist as singular data frames and/or data sets, or as multiple data frames and/or data sets in one or more layers. In examples, the formatted data may include meta-data that may provide additional annotation for machine learning models. In other examples, the formatted data may include dynamic data frames that may be scaled depending on models and applications. The formatted data 216 may exist in a myriad of formats, including, but not limited to, tab delineated formats, rich text formats, comma separated values, excel spreadsheets, XML files, html files, and/or other readable and/or writable data formats.

The formatted data 216 may be fed into the machine learning models 218, wherein the individual machine learning models 218 may be trained to identify gap(s) in care. The different machine learning models 218 may comprise different architectures and/or hyperparameters which may be compared to one another. For example, training a first machine learning model may include training one or more of the machine learning models 218 having different architectures and/or hyperparameters, and selecting the first machine learning model from the one or more machine learning models based at least in part on an accuracy metric, model complexity, and/or processing speed.

FIG. 2 depicts several machine learning models that may be used depending on the data structures and types of gap(s) in care being monitored. 218A depicts a multi-layer perceptron having an input layer comprising input nodes, a single hidden layer (intervening the input nodes and the output nodes), and an output layer comprising output nodes. In examples, the number of input nodes may equal the number of column vectors present in the data set, and the number of output nodes may be equal to the outcome of gap(s) in care (e.g., positive or negative). For example, the multi-layer perceptron may be trained to output the gap(s) in care for medication based on input data including laboratory values and/or medication data. 218B depicts a random forest algorithm that may take an initial data set and walk through decision tree(s) to arrive at a binary decision. 218N depicts a linear regression which may take a multi-dimensional dataset(s) and utilize linear regression to arrive at a potential gap in care. Additionally, the machine learning models may also comprise, for example, support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, and/or average one-dependence estimators (AODE), Bayesian belief network (BNN), and/or Bayesian networks), clustering algorithm(s) (e.g., k-means, k-medians, expectation maximization (EM)), hierarchical clustering, and/or neural network(s) (e.g., a multilayer perceptron (MLP), ResNet50, ResNet101, ResNet 152, VGG, DenseNet, PointNet).

FIG. 2 also depicts output data 220 that may be generated from the machine learning models 218. The output data 220 may include data regarding gap(s) in care (e.g., colonoscopy, mammogram, and/or untreated diabetes), as well as a probability and/or threshold value. Other output data 220 may include, for example, confidence values for point estimates, sensitivity, specificity, and/or model fit statistics (e.g., Akaike's information criterion, R values, and/or area under the curve). For example, 220A depicts output data 220 for a first gap in care, wherein the gap in care is a colonoscopy. The gap in care model may provide output data, recommended action(s) for care, and/or an indication as to whether the individual has insurance benefits (referred to as “Benefit for Care” in FIG. 2). In other examples, 220B depicts output data 220 for a second gap in care, wherein the gap in care is a mammogram. Similar to 220A, the gap in care model may provide output data, recommended action(s) for care, and/or an indication as to whether the individual has insurance benefits. Further, in another example, 220N depicts output data 200 for a gap in care, wherein the gap in care is untreated diabetes. Similar to 220A and 220B, the gap in care model may provide output data, recommended action(s) for care, and/or an indication as to whether the individual has insurance benefits.

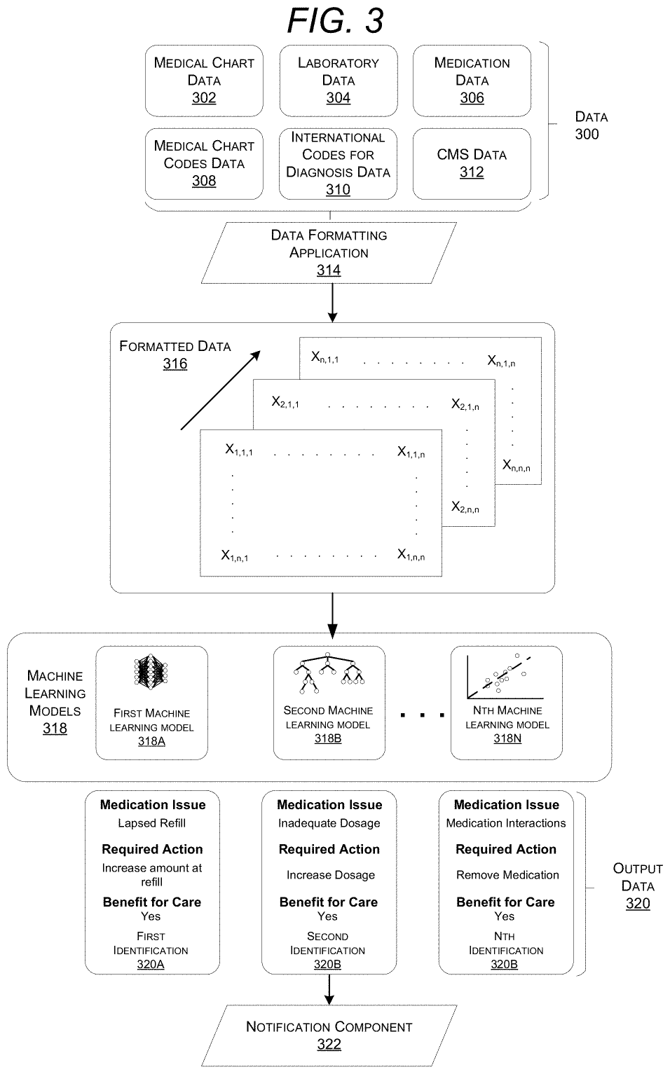

FIG. 3 illustrates a conceptual diagram of example components of surfacing recommended action(s) for medication architecture and systems. Data 300 from disparate sources may include at least one of medical chart data 302, laboratory data 304, medication data 306, medical chart codes 308, international codes for diagnosis data 310, and/or Centers for Medicare and Medicaid Services (CMS) data 312. The data may be fed into a data formatting application 314, where data may be formatted into model features configured to be input to the machine learning models. The formatted data 316 may be multi-dimensional to occupy N dimensional space and may be formatted to fit individual machine learning models. The formatted data may be fed into machine learning models 318A-318N (hereinafter referred to individually as “item 318A,” “item 318B,” etc., and generically as “item 318”), wherein the individual machine learning models may be trained to surface recommended action(s) for medication. The machine learning models may generate output data 320A-320N (herein after referred to individually as “item 320A”, “item 320B”, etc., and generically as “item 320). The output data 320 may include data regarding surfacing recommended action(s) for medication (e.g. identifying unfilled prescriptions, contraindicated polypharmacy), and additional data such including, but not limited to, probability and/or threshold data. The output data 320 may be sent to the notification component 322 which may transfer the notification from the remote computing resource 118 to the computing device 106 via the network 116 to display the notification to the medical service provider 102.

The medical chart data 302 may include at least one of, but not limited to, data regarding family health history, diet data, exercise, sexual history, smoking, alcohol consumption, immunization records, motor development, cognitive, intellectual, age, sex, race, religion, occupation, surgical history, obstetric history, allergies, hospital admissions, specialist consultations, and/or physical checkup visits. Laboratory data 204 may include at least one of, but not limited to, data regarding cell counts (e.g., red blood cell count, hematocrit, neutrophil count, lymphocyte count, monocyte count, eosinophil count, basophil count), cardiac markers (e.g., troponin I, troponin T and/or, myoglobin), general chemistries (e.g., albumin, alkaline phosphatase, anion gap, ammonia, ALT, AST, bilirubin, blood urea nitrogen, creatinine, creatine, calcium, chloride, carbon dioxide, ethanol, folic acid, glucose, HDL, LDL, iron, iron binding capacity, lactic acid, lactate, lipase, magnesium, osmolarity, parathyroid hormone, phosphorous, sodium, T3, T4, TSH, uric acid, and/or triglycerides), urine data (e.g., color, specific gravity, pH, sodium, potassium, protein, amylase, creatinine, and/or magnesium), and coagulation data (e.g., thrombin time, platelets, plasminogen, fibrinogen, and/or bleeding time). While this laboratory data is enumerated, other data may be more are complicated. Medication data 306 may include data regarding least one of, but not limited to, medication name, medication class, dosage, refill time(s), route(s) of administration, drug interaction(s), and/or contraindications. Medical chart codes data 308 may include data on at least one of, but not limited to, chart codes for tests, surgeries, evaluations, procedures, surgeries, and prescriptions. International codes for diagnosis (ICD) data 310 may include data regarding at least one, but not limited to, codes for diagnoses, symptoms, and procedures through the ICD coding system.

The data formatting application 314 may receive the data 300 that may be derived from multiple disparate data sources via the computing network 116. On its own, the data from the disparate sources is likely to exist in disparate formats. However, the data may be formatted into coherent data structures and formats in the data formatting application 314. These data structures may be multi-dimensional and may include associated meta-data that may be unpacked and formatted within the data structures. In examples, the disparate data sources may include annotations. The data structures may be formatted into model features that may be configured to be input to the machine learning models. Formatting data into model features may require several data manipulations. For example, data may be standardized and/or normalized to bring data with different scales into similar and/or the same scale(s). Data standardization is the process of rescaling features to have the properties of a Gaussian distribution where the mean is equal to zero and the standard deviation is equal to one. Data normalization is the process of rescaling the features such that the range of the data is fixed. For example, the range may be fixed between zero and one and normalized based on a sigmoid function. In other examples, the range may be fixed between zero and ten, and normalized based on a rectified linear unit function. While standardization and normalization approaches are discussed, other approaches may be utilized.

The data formatting application 314 may output formatted data 316. The formatted data 316 may be multi-dimensional and may exist in N dimensional space. The formatted data 316 may exist as singular data frames and/or data sets, or as multiple data frames and/or data sets in one or more layers. In examples, the formatted data may include meta-data that may provide additional annotation for machine learning models. In other examples, the formatted data may include dynamic data frames that may be scaled depending on models and applications. The formatted data 316 may exist in a myriad of formats, including, but not limited to, tab delineated formats, rich text formats, comma separated values, excel spreadsheets, XML files, html files, and/or other readable and/or writable data formats.

The formatted data 316 may be fed into machine learning models 318, wherein the individual machine learning models 318 may be trained to surface recommended action(s) for medication. The different machine learning models may comprise different architectures and/or hyperparameters which may be compared to one another. For example, training a first machine learning model may include training one or more of the machine learning models having different architectures and/or hyperparameters, and selecting the first machine learning model from among the one or more machine learning models based at least in part on an accuracy metric, model complexity, and/or processing speed.

FIG. 3 depicts several machine learning models that may be used depending on the data structures and types of surfacing recommended action(s) for medication. 318A depicts a multi-layer perceptron having an input layer comprising input nodes, a single hidden layer (intervening the input nodes and the output nodes), and an output layer comprising output nodes. In examples, the number of input nodes may equal the number of column vectors present in the data set, and the number of output nodes may be equal to the outcome of a medication interaction with another medication (e.g., positive or negative). For example, the multi-layer perceptron may be trained to output recommended action(s) for medication based on input data including laboratory values and/or medication data. 318B depicts a random forest algorithm that may take an initial data set and walk through decision tree(s) to arrive at increasing or decreasing a medication dosage. 318N depicts a linear regression which may take multi-dimensional dataset(s) and utilize linear regression to arrive at a recommended action(s) for medication. Additionally, the machine learning models may comprise, for example, a support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, average one-dependence estimators (AODE), Bayesian belief network (BNN), and/or Bayesian networks), clustering algorithm(s) (e.g., k-means, k-medians, and/or expectation maximization (EM)), hierarchical clustering, and/or neural network(s) (e.g., a multilayer perceptron (MLP), ResNet50, ResNet101, ResNet 152, VGG, DenseNet, and/or PointNet).

FIG. 3 also depicts output data 320 that may be generated from the machine learning models 318. The output data 320 may include data regarding recommended action(s) for medication (e.g., increasing prescription refill amount, lowering doses, and/or decreasing number of medications), as well as probability and/or threshold value(s). Other output data 320 may include, for example, confidence values for point estimates, sensitivity, specificity, and/or model fit statistics (e.g., Akaike's information criterion, R values, and/or area under the curve). For example, 320A depicts output data 320 for a first medication issue and recommended action for medication, wherein the medication issue is a lapsed refill. The model for surfacing recommended actions for medications may provide output data and/or recommended action(s) for adequately addressing the medication issue (e.g. increasing the amount of the refill to reduce refill frequency), and/or an indication as to whether the individual has insurance benefits (referred to as “Benefit for Care” in FIG. 3). In other examples, 320B depicts output data 320 for a medication issue and recommended action(s), wherein the medication issue is inadequate dosage. Similar to 320A, the model for surfacing recommended action(s) for medication(s) may provide output data and/or recommended action(s) for adequately addressing the medication issue (e.g. increasing the dosage of the medication), and/or an indication as to whether the individual has insurance benefits. Further, in another example, 320N depicts output data 300 for medication issues and recommended action, wherein the medication issue is medication interaction. Similar to 320A and 320B, the model for surfacing recommended actions for medication may provide output data, recommended action(s) for adequately addressing the medication issue (e.g. removing medication), and/or and an indication as to whether the individual has insurance benefits.

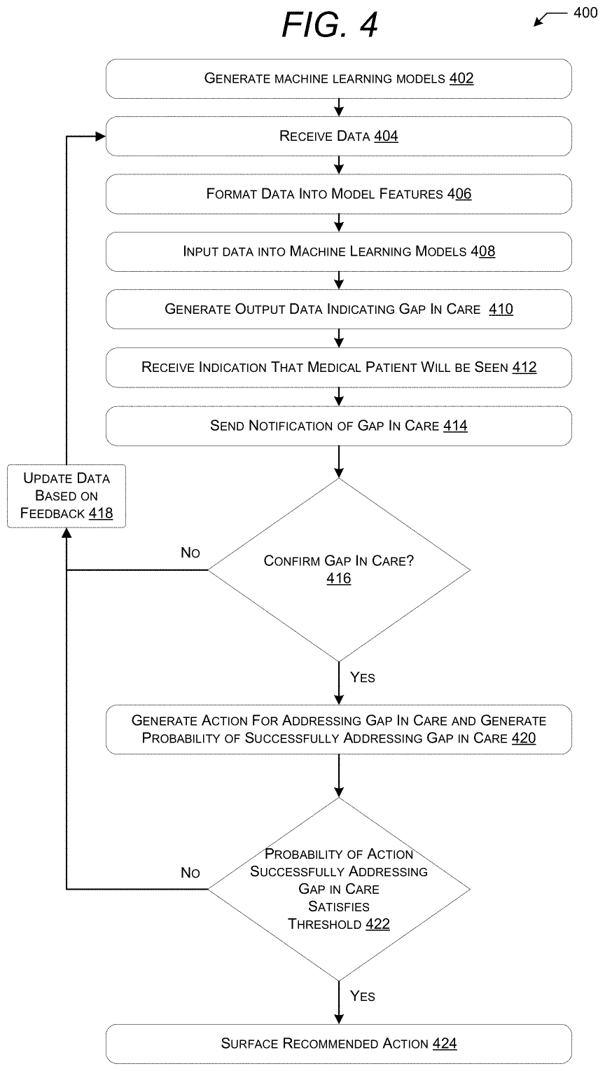

FIG. 4 illustrates a flow diagram of an example process for identifying gaps in care. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures, and systems described in the examples herein, such as, for example, those described with respect to FIGS. 1-3 and 5-7, although the processes may be implemented in a wide variety of other environments, architectures, and systems.

FIG. 4 illustrates a flow diagram of an example process 400 for identifying gaps in care. The order in which the operations or steps are described is not intended to be construed as a limitation, and any number of the described operations may be combined in any order and/or in parallel to implement process 400.

At block 402, the process 400 may include generating one or more machine learning models. For example, the machine learning models may be configured to identify one or more gaps in care and surface a recommendation for addressing the one or more gaps in care. The trained machine learning models may then be used to determine a likelihood that the recommendation for addressing the one or more gaps in care will be successfully implemented.

At block 404, the process 400 may include receiving data, wherein the data may consist of data from disparate sources received through a network and deposited into a database. The data used to train the machine learning models may be received into the database from multiple disparate data sources via a computing network. On their own, the data from the disparate sources may exist in disparate formats and may be formatted into coherent data structures and formats. These data structures may be multi-dimensional and may include associated meta-data that may be unpacked and formatted within the data structures.

At block 406, the process 400 may include formatting the data into model features. The disparate data sources may include annotations, which may require formatting. The data structures may be further formatted into model features that may be configured to be inputs to machine learning models. Formatting data into model features may require several data manipulations. For example, data may be standardized and/or normalized to bring data with different scales into a similar and/or the same scale(s).

At block 408, the process 400 may include inputting data into the machine learning models. The machine learning models may be individually trained to identify different gaps in care. The machine learning models may receive the inputted, formatted data and process that data utilizing machine learning approaches. Additionally, the machine learning models may comprise, for example, support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, average one-dependence estimators (AODE), Bayesian belief network (BNN), and/or Bayesian networks), clustering algorithm(s) (e.g., k-means, k-medians, and/or expectation maximization (EM)), hierarchical clustering, and/or neural network(s) (e.g., a multilayer perceptron (MLP), ResNet50, ResNet101, ResNet 152, VGG, DenseNet, and/or PointNet).

At block 410, the process may include generating output data indicating a gap in care. The output data may include at least one of, for example, a gap in care, a recommendation for addressing the gap in care, a probability that the recommendation with successfully address the gap in care, a threshold, and/or data regarding whether the recommendation is covered by an insurance benefit. Other output data may include sensitivity data, specificity data, Akaike's information criterion, area under the curve, and/or other measures of model fit and classification accuracy.

At block 412, the process 400 may include receiving an indication that a medical patient will be seen. The indication may be based upon data related to a medical schedule, calendar, and/or appointment in medical records. In other examples, the indication may be based upon data that suggests the medical patient may be seen, including, but not limited to, time frames for diagnostic follow-ups, tests, annual check-ups, and/or medication refills.

At block 414, the process 400 may include sending a notification of a gap in care to a user. The user, in examples, may be a medical service provider. This notification may surface on the user's device and/or apparatus in an electronic medical chart, database, or other system.

At block 416, the gap in care may be confirmed or rejected. If the gap in care is rejected, the systems and methods may proceed to block 418, wherein the process 400 may update based on feedback data. If the if the gap in care is confirmed, the systems and methods may proceed to block 420, generating an action for addressing the gap in care and/or generating a probability that the action will successfully address the gap in care.

At block 418, the process 400 may include updating data based on feedback. In examples the feedback may be generated from block 416 wherein the gap in care may be rejected. In examples, the feedback data may be generated from block 422 where the probability of an action successfully addressing a gap in care does not satisfy a threshold. The feedback data may be fed back into the model, updating and/or improving the model.

At block 420, the process 400 may include generating actions for addressing one or more gaps in care and generating a probability of successfully addressing the one or more gaps in care. The probability may also include data regarding the confidence of the probability estimate and data regarding a threshold associated with the probability.

At bock 422, the process 400 may include determining if the probability of a generated action to successfully address a gap in care satisfies a threshold. If the probability of the action addressing the gap in care satisfies the threshold, the procedure may continue to block 424. If the probability of the action addressing the gap in care fails to satisfy the threshold, the systems and methods may update based on feedback data 418 that the recommended action failed to satisfy the threshold.

At block 424, the process 400 may include surfacing the recommended action to a medical service provider. For example, a command may be sent to a device associated with the medical service provider, where the command may cause an application associated with the device to initiate and/or cause display of a visual indication of the recommended action.

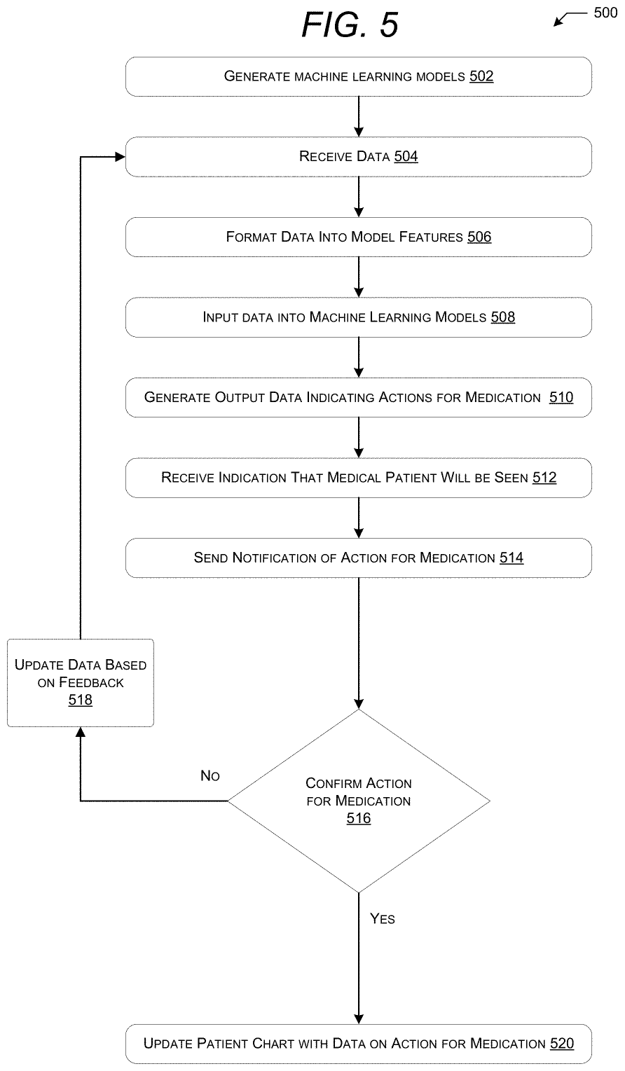

FIG. 5 illustrates a flow diagram of an example process surfacing recommended actions for medication. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures, and systems described in the examples herein, such as, for example those described with respect to FIGS. 1-4 and 6-7, although the processes may be implemented in a wide variety of other environments, architectures, and/or systems.

FIG. 5 illustrates a flow diagram of an example process 500 for surfacing recommended actions for medications. The order in which the operations or steps are described is not intended to be construed as a limitation, and any number of the described operations may be combined in any order and/or in parallel to implement process 500.

At block 502, the process 500 may include generating one or more machine learning models. For example, the machine learning models may be configured to recommend an action for medication. The trained machine learning models may be used to determine a likelihood that the recommendation action for medication will be successfully implemented.

At block 504, the process 500 may include receiving data, wherein the data may consist of data from disparate sources received through a network and deposited into a database. The data used to train the machine learning models may be received into a database from multiple disparate data sources via a computing network. On their own, the data from the disparate sources may exist in disparate formats and may be formatted into coherent data structures and formats. These data structures may be multi-dimensional and may include associated meta-data that may be unpacked and/or formatted within the data structures.

At block 506, the process 500 may include formatting the data into model features. The disparate data sources may include annotations and may require formatting. The data structures may be formatted into model features that may be configured to be input to machine learning models. Formatting data into model features may require several data manipulations. For example, data may be standardized and/or normalized to bring data with different scales into similar and/or the same scale(s). New data may also be generated appropriate scales using other techniques appropriate for machine learning models.

At block 508, the process 500 may include inputting the data into machine learning models. The machine learning models may be individually trained to identify different recommended actions for medication. The machine learning models may receive the inputted and formatted data, and process the data utilizing machine learning approaches. Additionally, the machine learning models may comprise, for example, support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, average one-dependence estimators (AODE), Bayesian belief network (BNN), and/or Bayesian networks), clustering algorithm(s) (e.g., k-means, k-medians, and/or expectation maximization (EM)), hierarchical clustering, and/or neural network(s) (e.g., a multilayer perceptron (MLP), ResNet50, ResNet101, ResNet 152, VGG, DenseNet, and/or PointNet).

At block 510, the process 500 may include generating output data indicating actions for medication. The output data may include at least a recommended action for medication, a probability that the recommendation will successfully address the gap in care, a threshold, and/or data regarding whether the recommendation is covered by an insurance benefit. Other output data may include sensitivity data, specificity data, Akaike's information criterion, area under the curve, and/or other measures of model fit and classification accuracy.

At block 512, the process 500 may include receiving an indication that a medical patient will be seen. The indication may be based upon data related to a medical schedule, calendar, and/or appointment in medical records. In examples, the indication may also be based upon data the suggests a medical patient may be seen, including, but not limited to, time frames for diagnostic follow-ups, tests, annual check-ups, and/or medication refills.

At block 514, the process 500 may include sending a notification of a gap in care to a user. The user, in examples, may be a medical service provider. This notification may surface on the user's device and/or apparatus in an electronic medical chart, database, or other system.

At block 516, the process 500 may include deciding on an action for medication. In examples, the action for medication may be confirmed or rejected. If the action for medication is rejected, the systems and methods may proceed to block 518, wherein the process 500 may update based on feedback data. If the if the action for medication is confirmed, then the process may proceed to block 520.

At block 518, the process 500 may include updating data based on feedback. In examples, the feedback may be generated from block 516 wherein the action for medication may be rejected. This feedback data may be fed back into the model to update and/or improve the model.

At block 520, the process 500 may include updating the patient chart with data regarding actions for medication. The data may include, but is not limited to, changes to refill amounts, refill schedules, dosages, removal of current prescriptions, location of pharmacy pick-ups, and/or changes to medication brand.

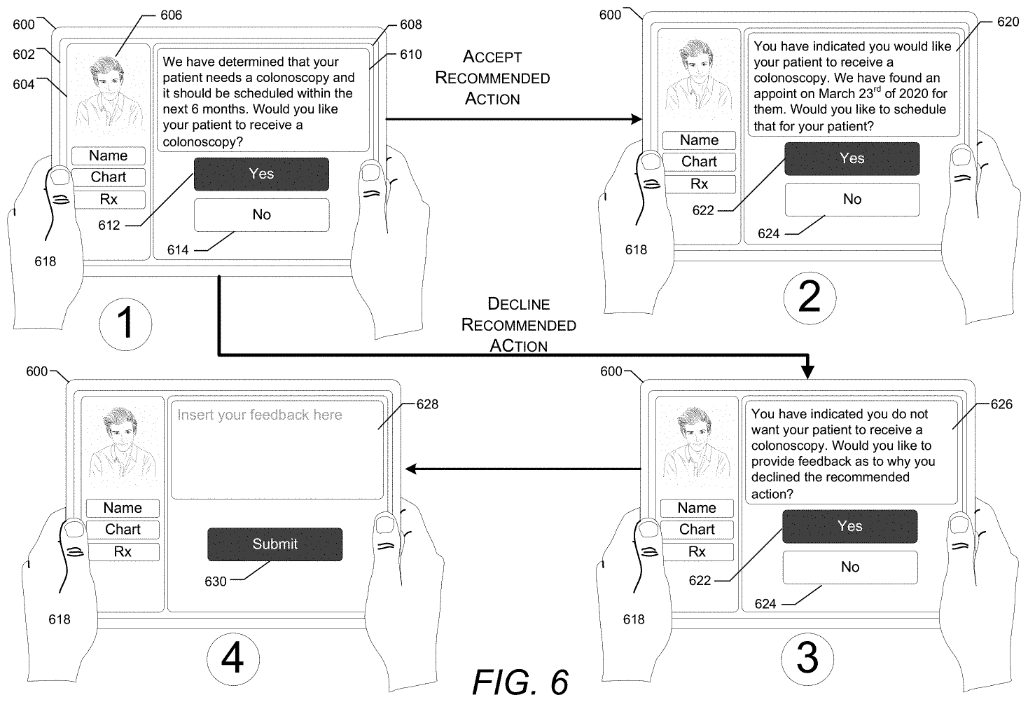

FIG. 6 illustrates an example user interface utilized for confirming or rejecting a gap in care utilizing the machine learning models described herein. The decision tree bifurcation shown in FIG. 6 is illustrated by arrows. The device 600, which may be a computing device, may include a display 602 having a first area 604 and a second area 608. For instance, the first area 604 may include an image of a patient 606 along with the patent's name, medical chart, and/or prescription information. While FIG. 6 illustrates certain background information, other information may be displayed and/or presented differently than as shown. The background information may be accessed by a user 618 interacting with the display 602. For instance, the user 618 may select “Chart” within the first area, thus presenting the medical charts of the patient 606 on the display 602.

At step 1, the second area 608 may display a first prompt within a first field 610 that relates to a gap in care and a recommended action. The gap in care and recommended action may be received from the notification component 138 and 222. The user 618 may respond to the first prompt by selecting a button to accept/confirm the recommended action 612 or by selecting a button to reject/decline the recommended action 614.

At step 2, if the recommended action is accepted/confirmed, the device 600 may proceed to populate the second area 608 with a new prompt 620, notifying the user 618 that the recommended action was confirmed and the profile was updated. In examples, the second area 608 may ask the user 618 if they would like to schedule the recommended action. By selecting the “Yes” button 622, the user 618 may select to proceed with scheduling the recommended action. By selecting the “No” button 624, the user 618 may end the process.

At step 3, if the initial gap in care and recommended action is rejected, then the device 600 may proceed to populate a second area 626 with a new prompt, notifying the user 618 that the gap in care and recommended action was rejected/declined and the profile was updated. In examples, the second area 626 may ask the user 618 if they would like to provide feedback on the reason for rejecting/declining the gap in care and recommended action.

At step 4, the user 618 may be presented with an input field 628 to provide feedback. In examples, the user 618 may choose to provide feedback regarding the user's 618 decision to decline recommended action. The user 618 may end the process by selecting the “No” button 624.

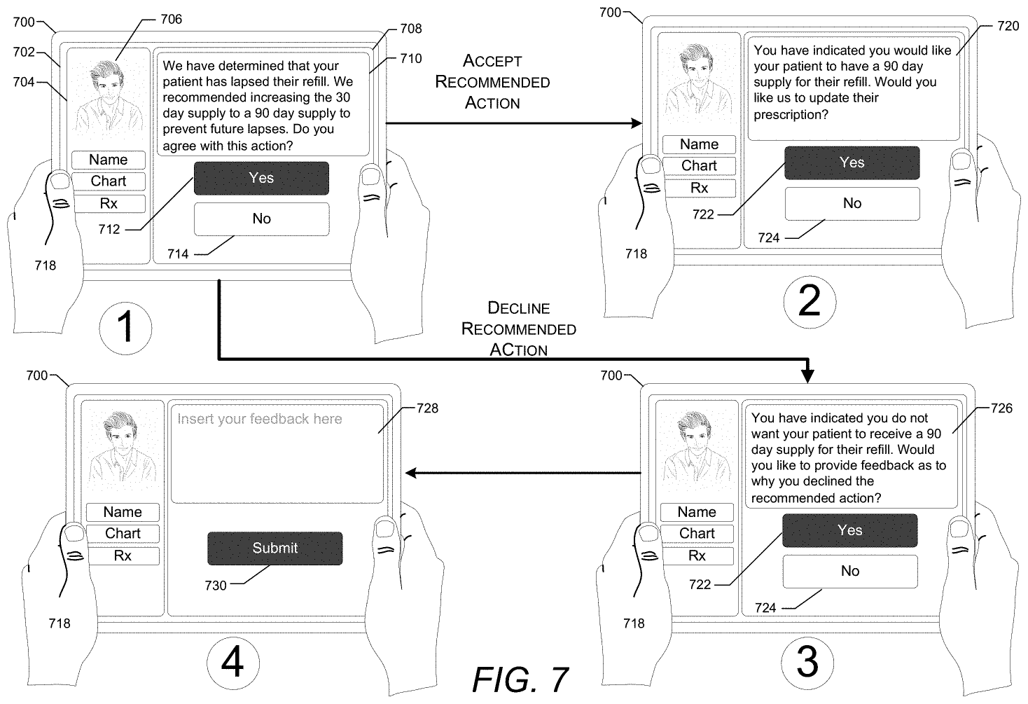

FIG. 7 illustrates an example process for confirming or rejecting a recommended action for medication. The decision tree bifurcation shown in FIG. 7 is illustrated by arrows. The device 700, which may be a computing device, may include a display 702 having a first area 704 and a second area 708. For instance, the first area 704 may include an image of a patient 706 along with the patient's name, medical chart, and/or prescription information. While FIG. 7 illustrates certain background information, other information may be displayed and/or presented differently than as shown. The background information may be accessed through a user 718 interacting with the display 702. For instance, the user 718 may select “Chart” within the first area and medical charts of the patient may be displayed on the display 702.

At step 1, the second area 708 may display a first prompt within a first field 710 that relates to a medication issue and a recommended action. The notification components 138 and 222 may receive the medication action and/or recommended action. The user 718 may respond to the first prompt by selecting a button to accept/confirm the recommended action 712 or by selecting a button to reject/decline the recommended action 714.

At step 2, if the recommended action is accepted/confirmed, the device 700 may proceed to populate the second area 708 with a new prompt 720, notifying the user 718 that the recommended action was confirmed and the profile was updated. In examples, the second area 708 may ask the user 718 if they would like to take the recommended action. By selecting the “Yes” button 722, the user 718 may select to proceed with taking the recommended action. By selecting the “No” button 724, the user 718 may end the process.

At step 3, if the initial medication issue and recommended action is rejected, then the device 700 may proceed to populate a different second area 726 with a new prompt 726, notifying the user 718 that the medication issue and recommended action was rejected/declined and the profile was updated. In examples, the second area 726 may ask the user 718 if they would like to provide feedback on the reason for rejecting/declining the medication issue and recommended action.

At step 4, the user 718 may be presented with an input field 728 to provide feedback. In examples, the user 718 may choose to provide feedback regarding the user's 718 decision to decline recommended action. The user 718 may end the process by selecting the “No” button 724.

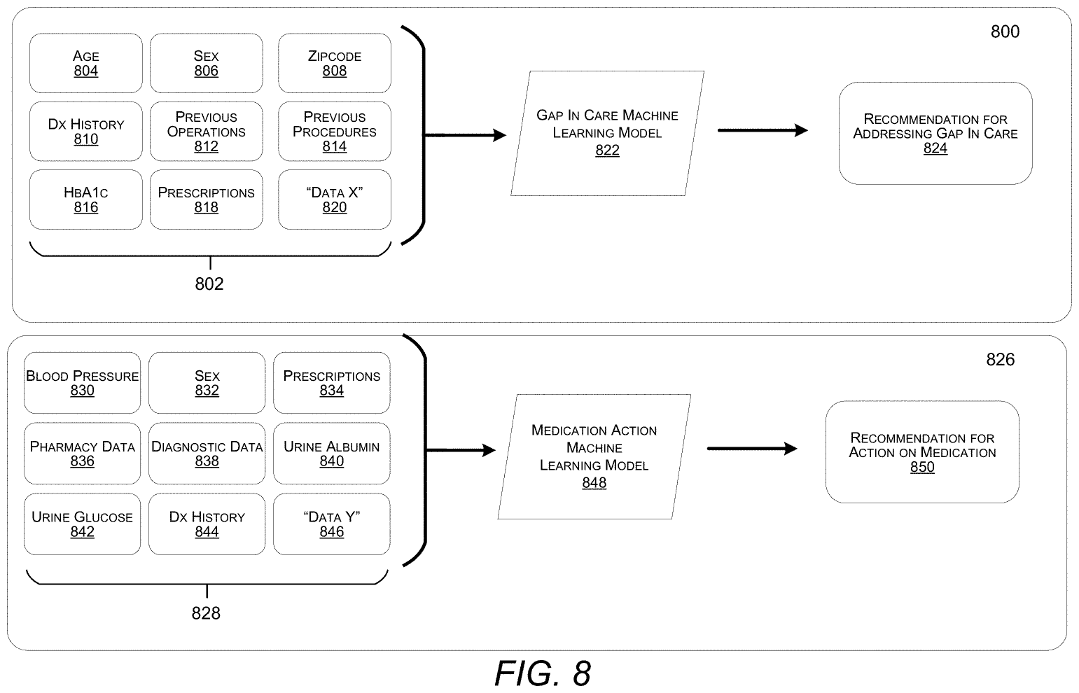

FIG. 8 illustrates a conceptual diagram of the components of input data, machine learning models, and output data associated with identifying gaps in care and surfacing recommend action(s) for medication. The environment 800 may describe the process and space in which a machine learning model 822 may receive input data 802 and generate recommendations for addressing gaps in care 824. The input data may be prioritized for the gap in care machine learning model 822 through a priori knowledge or prioritization strategies based on predefined criteria or through machine learning techniques to harvest data. A priori knowledge or prioritization may be based on clinical knowledge. In examples, age 804 may provide prognostic information indicating the need for colorectal cancer screening. However, age may be used for other gaps in care as well. Other data that may be determined a priori by predefined criteria may include, but is not limited to, sex 806, zip code 808, diagnosis (Dx) history 810, previous operations 812, previous procedures 814, glycated hemoglobin (HbA1c) 816, and/or prescriptions 818. In examples, data may be selected based on machine learning techniques. In examples, machine learning techniques may identify “data X” 520 as an important feature for determining a gap in care, wherein “data X” may be familial history of a disease, genetic data, BMI data, and/or other data. In another example, environment 826 may describe the process and space in which a machine learning model 848 may receive input data 828 and generate recommendations for medication actions(s) 850. The input data may be prioritized for the medication action machine learning model 548 through a priori knowledge or prioritization strategies based on predefined criteria or through machine learning techniques to harvest data. In examples, blood pressure 830 may provide information indicating a certain medication such as an angiotensin converting enzyme inhibitor. However, blood pressure may also be indicative of other diseases that may require different medication(s). Thus, it may be used as other data to determine a medication issue and recommended action. Other data that may be determined a priori by predefined criteria may include, but is not limited to, sex 832, prescriptions 834, pharmacy data 836, diagnostic data 838, urine albumin 840, urine glucose 842, and/or diagnostic history 844. Additionally, or alternatively, data may be selected based on machine learning techniques. In examples, machine learning techniques may identify “data y” 546 as an important feature for determining a medication issue and recommended action, wherein “data y” may be genetic markers that provide insights into pharmacogenomics and medication stratification.

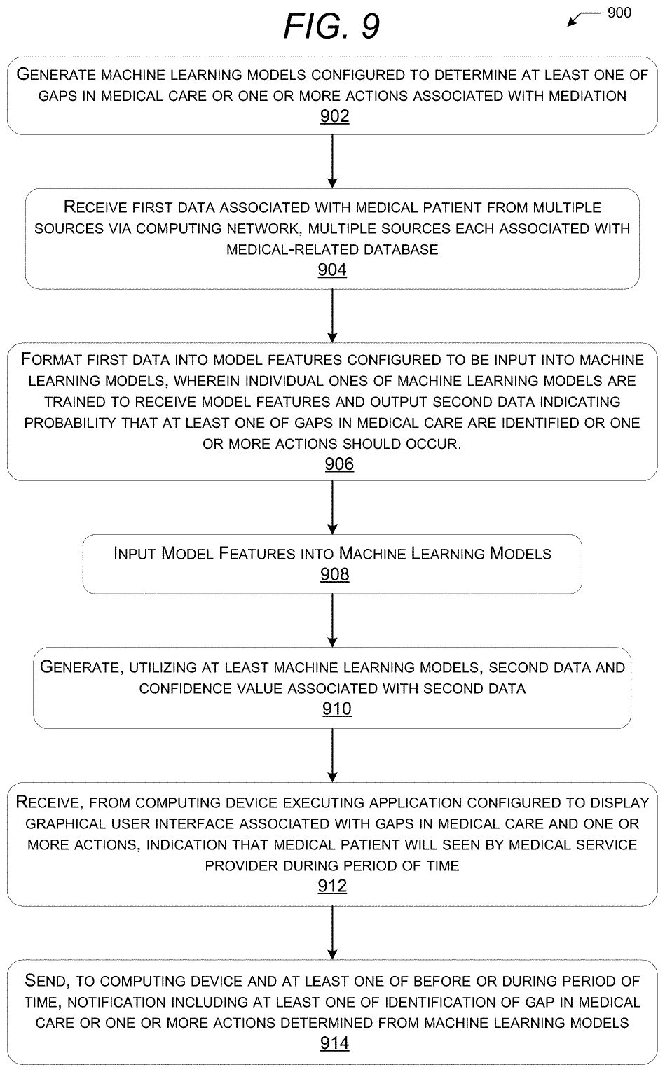

FIG. 9 illustrates a flow diagram of an example process for utilizing machine learning models for identifying gaps in care. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures, and systems described in the examples herein, such as, for example those described with respect to FIGS. 1-8 and 10, although the processes may be implemented in a wide variety of other environments, architectures, and/or systems.

FIG. 9 illustrates a flow diagram of an example process 900 for utilizing machine leaning models for identifying gaps in care. The order in which the operations or steps are described is not intended to be construed as a limitation, and any number of the described operations may be combined in any order and/or in parallel to implement process 900.

At block 902, the process 900 may include generating machine learning models configured to determine at least one of gaps in medical care or one or more actions associated with medication. The individual machine learning models may be trained to determine gaps in care for different potential types of care. The trained machine learning models may be used to determine a likelihood that the recommendation for addressing the one or more gaps in care will be successfully implemented.

At block 904, the process 900 may include receiving first data associated with a medical patient from multiple sources via a computing network, the multiple sources each associated with medical-related database. The data used to train the machine learning models may be received at the medical-related database from multiple disparate data sources via a computing network. On their own, the data from the disparate sources may exist in disparate formats. However, the data may be formatted into coherent data structures and formats. These data structures may be multi-dimensional and may include associated meta-data that may be unpacked and formatted within the data structures.

At block 906, the process 900 may include formatting the first data into model features configured to be input to the machine learning models, wherein individual ones of the machine leaning models are trained to receive the model features and output second data indicating a probability that the at least one of the gaps in medical care are identified or the one or more actions should occur. The data structures are further formatted into model features that may be configured to be input to the machine learning models. Formatting data into model features may require several data manipulations. For example, data may be standardized and/or normalized to bring data with different scales into similar or the same scale(s).

At block 908, the process 900 may include inputting the model features into the machine learning models. The machine learning models may be individually trained to identify different gaps in care. The machine learning models may receive the inputted, formatted data and process the data utilizing machine learning approaches. The machine learning approaches may comprise support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, average one-dependence estimators (AODE), Bayesian belief network (BNN), and/or Bayesian networks), clustering algorithm(s) (e.g., k-means, k-medians, and/or expectation maximization (EM)), hierarchical clustering, and/or neural network(s) (e.g., a multilayer perceptron (MLP), ResNet50, ResNet101, ResNet 152, VGG, DenseNet, and/or PointNet).

At block 910, the process 900 may include generating, utilizing at least the machine learning models, the second data and a confidence value associated with the second data. In examples, the machine learning models may be configured to identify gaps in care and recommend action(s), wherein individual machine learning models are trained to identify gaps in care, recommend action(s), and generate a probability that the recommended action(s) will address the gaps in care. The models may be developed and trained for gaps in care and may have overlapping features with other models. The selected features, normalization, standardization, and/or transformation of data may be similar across different disease focused models. In other examples, the models may be entirely independent data vectors. In examples, the machine learning models may determine a gap in care, based at least in part on machine learning techniques, which may be derived at least in part from the data, may recommend an action, may generate a probability for the recommended action, and may determine if the probability satisfies a threshold. The output data, including the gap in care and recommended action may then sent by a first computing device executing a first application and may be received by a second computing device executing a second application.

The machine learning models may utilize supervised and/or unsupervised learning. The machine learning models may include one or more linear discriminant analysis, classification and regression tree(s), decision tree learning, random forest model(s), nearest neighbor(s), support vector machine(s), logistic regression, general linear model(s), Bayesian model(s), and/or neural network(s).

At block 912, the process 900 may include receiving, from a computing device executing an application configured to display a graphical user interface associated with the gaps in medical care and the one or more actions, an indication that the medical patient will be seen by a medical care provider during a period of time. The medical service provider may be, for example, a nurse practitioner or a medical service provider's assistant.

At block 914, the process 900 may include sending, to a computing device and at least one of before or during the period of time, a notification including at least one of identification of the gap in medical care or the one or more actions as determined from the machine learning models. The notification may surface gaps in care on a device 106, 600, or 700, wherein the medical service provider may review the patient's chart, medical history, and/or other salient information. In examples, the medical service provider may confirm or reject the recommended action(s) for gas in care.

The process 900 may include user data that relates at least, but is not limited to, the health, socioeconomic status, geographic location, genetic history, and/or behavior of the user. The data may include, but is not limited to, medical records, chart codes, Centers for Medicare & Medicaid Services data, International Codes for Diagnosis data, medication data, diagnostic imaging data, and/or laboratory data.

The process 900 may be based at least in part on machine learning models. The machine learning models may determine that the medical patient is a member of a service, or that that the medical patient will be seen by a medical service provider at a specified time. Additionally, or alternatively, the process 900 may include prioritizing data to train the machine learning models. The prioritizing may be based on predefined criteria. The predefined criteria may include, but is not limited to, age, gender, diagnosis, medication use data, and/or previous procedure history. The prioritization may be based on machine learning approaches. Prioritization may include, but is not limited to, determining the impact specific data types may have on output data, determining that the impact satisfies a threshold impact, and prioritizing the data type.

The process 900 may include incorporating feedback data into the machine learning techniques. The feedback may include, but it not limited to, indications of whether the identified gap(s) in care and/or recommended action(s) were correct. The feedback may be used to adjust probabilities and/or thresholds of the models. The feedback may be received over a period of time and fed into the machine learning models continuously, or at regular or irregular intervals. The machine learning models may be updated continuously, or at regular or irregular intervals.

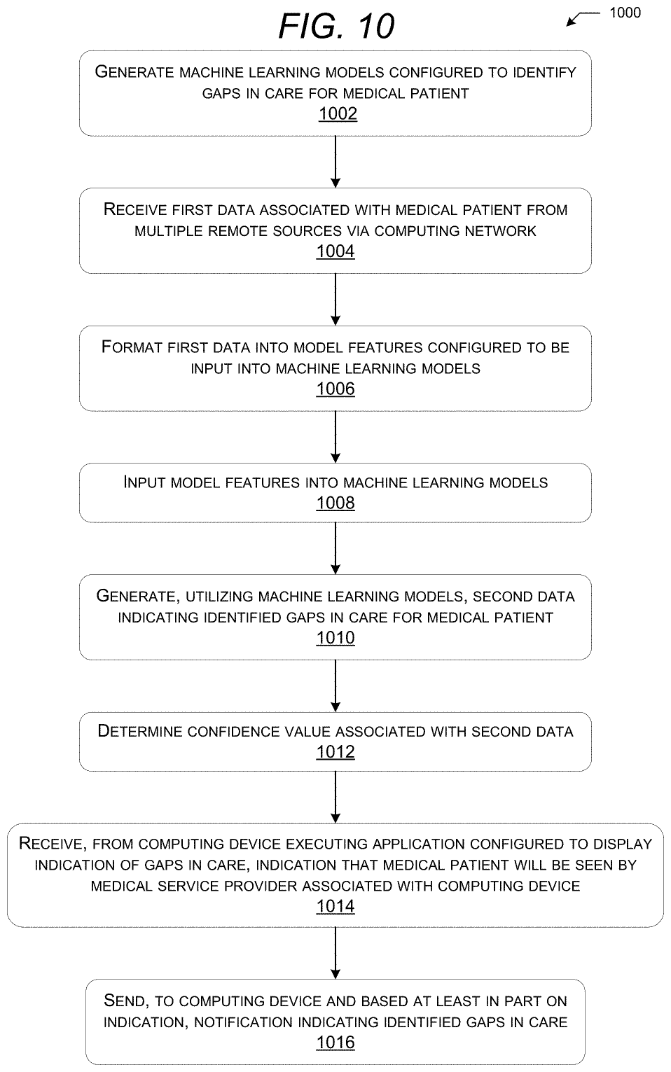

FIG. 10 illustrates a flow diagram of an example process for utilizing machine learning models for surfacing recommended actions for medication. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures, and systems described in the examples herein, such as, for example those described with respect to FIGS. 1-9, although the processes may be implemented in a wide variety of other environments, architectures, and/or systems.

FIG. 10 illustrates a flow diagram of an example process 1000 for utilizing machine leaning models for surfacing recommended actions for medication. The order in which the operations or steps are described is not intended to be construed as a limitation, and any number of the described operations may be combined in any order and/or in parallel to implement process 1000.

At block 1002, the process 1000 may include generating machine learning models configured to identify gaps in care for a medical patient. The individual machine learning models may be trained to surface recommended action(s) for different medication. The trained machine learning models may be used to determine a likelihood that the recommended action(s) for medication will be successfully implemented

At block 1004, the process 1000 may include receiving first data associated with the medical patient from multiple remote sources via a computing network. The data used to train the machine learning models may be received at the medical-related database from multiple disparate data sources via a computing network. On their own, the data from the disparate sources may exist in disparate formats. However, the data may be formatted into coherent data structures and formats. These data structures may be multi-dimensional and may include associated meta-data that may be unpacked and formatted within the data structures.

At block 1006, the process 1000 may include formatting the first data into model features configured to be input to the machine learning models. The disparate data sources may include annotations. The data structures are further formatted into model features that may be configured to be input to the machine learning models. Formatting data into model features may require several data manipulations. For example, data may be standardized and/or normalized to bring data with different scales into a similar and/or the same scale(s).

At block 1008, the process 1000 may include inputting the model features into the machine learning models. The machine learning models may be individually trained to surface recommended actions for different medications or action(s) for medication. The machine learning may receive the inputted, formatted data and process the data utilizing machine learning approaches. The machine learning approaches may comprise support vector machine(s) (SVM) (e.g., Nystroem Kernel SVM and/or radial basis function (RBF) kernel SVM), regression algorithm(s) (e.g., ordinary least squares regression (OLSR), linear regression, logistic regression, Ridge regression, Lasso regression, and/or ElasticNet regression), decision tree algorithm(s) (e.g., classification and regression tree (CART), iterative dichotomiser 3 (ID3), Chi-squared automatic interaction detection (CHAID), decision stump, conditional decision trees, LightGBM, gradient-boosting machines (GBM), and/or gradient boosted regression trees (GBRT)), Bayesian algorithm(s) (e.g., naïve Bayes, Gaussian naïve Bayes, multinomial naïve Bayes, average one-dependence estimators (AODE), Bayesian belief network (BNN), and/or Bayesian networks), clustering algorithm(s) (e.g., k-means, k-medians, and/or expectation maximization (EM)), hierarchical clustering, and/or neural network(s) (e.g., a multilayer perceptron (MLP), ResNet50, ResNet101, ResNet 152, VGG, DenseNet, and/or PointNet).

At block 1010, the process 1000 may include generating, utilizing the machine learning models, second data indicating identified gaps in care for the medical patient. In examples, the machine learning models may be configured to surface recommended action(s), wherein individual machine learning models are trained to identify potential issues with the medication, recommend an action, and generate a probability that the recommended action will address the issue with the medication. The models for identifying gaps in care may have overlapping and/or independent features with models for surfacing recommended actions for medications. The selected features, normalization, standardization, and transformation of data may be similar across different disease focused models. In other examples, the models may be entirely independent data vectors. In this example, the machine learning models may determine a medication issue, based at least in part on machine learning techniques, which may be derived at least in part from the data, may recommend an action, may generate a probability for the recommended action, and may determine if the probability satisfies a threshold. The output data, including the gap in care and recommended action may then sent by a first computing device executing a first application and may be received by a second computing device executing a second application.

At block 1012, the process 1000 may include determining a confidence value associated with the second data. In examples, the machine learning modes may be configured to identify at least one gap in care and recommend action(s), wherein individual machine learning models are trained to identify gap(s) in care, recommend action(s), and generate a probability that the recommended action will address the gap(s) in care.

At block 1014, the process 1000 may include receiving, from a computing device executing an application configured to display indications of the gaps in care, an indication that the medical patient will be seen by a medical service provider associated with the computing device.

At block 1016, the process 1000 may include sending, to the computing device and based at least in part on the indication, a notification indicating the identified gaps in care. The notification may surface a recommended action a device 106, 600, or 700, wherein the medical service provider may review the patient's chart, medical history, and/or other salient information, and may confirm or reject the recommended action(s) for medication.

The process 1000 may include user data that relates at least, but is not limited to, the health, socioeconomic status, geographic location, genetic history, and/or behaviors of the user. The data may include, but is not limited to, medical records, chart codes, Centers for Medicare & Medicaid Services data, International Codes for Diagnosis data, medication data, diagnostic imaging data, and/or laboratory data.

The process 1000 may include at least one machine learning model. The machine learning model(s) may utilize supervised or unsupervised learning. The machine learning model(s) may include one or more of linear discriminant analysis, classification and regression tree(s), decision tree learning, random forest model(s), nearest neighbor(s), support vector machine(s), logistic regression, general linear model(s), Bayesian model(s), and/or neural network(s).

The process 1000 may be based at least in part on machine learning models. The machine learning models may determine that the medical patient is a member of a service, or that the medical patient will be seen by a medical service provider at a given time. Additionally, or alternatively, the process 1000 may include prioritizing data prior to train the machine learning models. The prioritizing may be based on predefined criteria. The predefined criteria may include, but is not limited to, age, gender, diagnosis, medication use data, and/or previous procedure history. The prioritization may be based on machine learning approaches. Prioritization may include, but is not limited to, determining the impact specific data types may have on output data, determining that the impact satisfies a threshold impact, and prioritizing the data type.

The process 1000 may include incorporating feedback data into the machine learning techniques. The feedback may include, but is not limited to, indications of whether the identified medication issue(s) and recommended action(s) were correct. The feedback may be used to adjust probabilities and/or thresholds of the models. The feedback may be received over a period of time and fed into the machine learning models continuously, or at regular or irregular intervals. The machine learning models may be updated continuously, or at regular or irregular intervals.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

What is claimed is:

1. A system comprising:
one or more processors; and
non-transitory computer-readable media storing first computer-executable instructions that, when executed by the one or more processors, cause the one or more processors to perform operations comprising:
generating machine learning models configured determine at least one of gaps in medical care or one or more actions associated with medication;
receiving first data associated with a medical patient from multiple sources via a computing network, the multiple sources each associated with medical-related database;
formatting the first data into model features configured to be input into the machine learning models, wherein individual ones of the machine learning models are trained to receive the model features and output second data indicating a probability that the at least one of the gaps in medical care are identified or the one or more actions should occur;
inputting the model features into the machine learning models;
generating, utilizing at least the machine learning models, the second data and a confidence value associated with the second data;
receiving, from a computing device executing an application configured to display a graphical user interface associated with the gaps in medical care and the one or more actions, an indication that the medical patient will be seen by a medical service provider during a period of time; and
sending, to the computing device and at least one of before or during the period of time, a notification including at least one of identification of the gap in medical care or the one or more actions as determined from the machine learning models.

2. The system of claim 1, wherein the first data includes at least one of medical records, chart codes, Centers for Medicare & Medicaid Services data, International Codes for Diagnosis data, pharmacy data, medication data, or laboratory data.

3. The system of claim 1, the operations further comprising:
identifying an event including at least one of a missed appointment or an interval of time without a medical-related appointment;
determining, utilizing at least one of the machine learning models, a probability for successfully addressing at least one of the gaps in medical care or the one or more actions, the probability associated with the event; and
wherein sending the notification comprises sending the notification based at least in part on the probability satisfying a threshold probability for successfully addressing the at least one of the haps in medical care or the one or more actions.

4. The system of claim 1, the operations further comprising weighting the first data prior to training the machine learning models, the weighting based at least in part on at least one of an age of the medical patient, a gender of the medical patient, diagnoses associated with the medical patient, medication use data associated with the medical patient, or procedure history associated with the medical patient.

5. A method comprising:
generating machine learning models configured to identify gaps in care for a medical patient;
receiving first data associated with the medical patent from multiple remote sources via a computing network;
formatting the first data into model features configured to be input into the machine learning models;
inputting the model features into the machine learning models;
generating, utilizing the machine learning models, second data indicating identified gaps in care for the medical patient;
determining a confidence value associated with the second data;
receiving, from a computing device executing an application configured to display indications of gaps in care, an indication that the medical patient will be seen by a medical service provider associated with the computing device; and
sending, to the computing device and based at least in part on the indication, a notification indicating the identified gaps in care.

6. The method of claim 5, wherein the first data includes at least one of medical records, chart codes, Centers for Medicare & Medicaid Services data, International Codes for Diagnosis data, pharmacy data, medication data, or laboratory data.

7. The method of claim 5, further comprising:
identifying an event including at least one of a missed appointment or an interval of time without a medical-related appointment;
determining, utilizing at least one of the machine learning models, a probability for successfully addressing the identified gaps in care, the probability associated with the event; and
wherein sending the notification comprises sending the notification based at least in part on the probability satisfying a threshold probability for successfully addressing the identified gaps in care.

8. The method of claim 5, wherein the machine learning models include at least one of linear discriminant analysis, classification and regression tree, decision tree learning, random forest model, nearest neighbor, support vector machine, logistic regression, general linear model, Bayesian model, or neural network.

9. The method of claim 5, further comprising:
determining that the medical patient is a member of a predefined insurance service;
determining a procedure associated with the identified gaps in care;
generate a pre-approval for the procedure based at least in part on the medical patient being the member of the predefined insurance service; and
wherein the notification indicates the pre-approval.

10. The method of claim 5, further comprising weighting the first data prior to training the machine learning models, the weighting based at least in part on at least one of an age of the medical patient, a gender of the medical patient, diagnoses associated with the medical patient, medication use data associated with the medical patient, or procedure history associated with the medical patient.

11. The method of claim 5, further comprising:
determining an impact of a data type on the confidence value;
determining that the impact satisfies a threshold impact; and
weighting the data type based at least in part on the impact satisfying the threshold impact.

12. The method of claim 5, further comprising:
receiving feedback data associated with the notification;
determining, utilizing the feedback data and the machine learning models, a data type that impacts identification of the gap in care; and
updating the machine learning models to determine the gap in care based at least in part on the data type.

13. A system comprising:
one or more processors; and
non-transitory computer-readable media storing instructions that, when executed by the one or more processors, cause the one or more processors to perform operations comprising:
generating machine learning models configured to identify one or more actions associated with medication for a medical patient;
receiving first data from multiple sources via a computing network;
formatting the first data into model features configured to be input into the machine learning models;
inputting the model features into the machine learning models;
generating, utilizing the machine learning models, second data indicating the one or more actions;
assigning a confidence value to the second data;
receiving, from a computing device executing an application, an indication that the medical patient will be seen by a medical service provider; and
sending, to the computing device and based at least in part on the confidence value, a notification including the one or more actions.

14. The system of claim 13, wherein the first data includes at least one of medical records, chart codes, Centers for Medicare & Medicaid Services data, International Codes for Diagnosis data, pharmacy data, medication data, or laboratory data.

15. The system of claim 13, the operations further comprising:
determining third data associated with the medical patient, the third data representing at least one of pharmacy information, dosing strategies, access to medication, or measures of polypharmacy;
determining a probability for successful implementation of the one or more actions based at least in part on the third data; and
wherein sending the notification comprises sending the notification based at least in part on the probability satisfying a threshold probability.

16. The system of claim 13, wherein the machine learning models include at least one of linear discriminant analysis, classification and regression tree, decision tree learning, random forest model, nearest neighbor, support vector machine, logistic regression, general linear model, Bayesian model, or neural network.

17. The system of claim 13, the operations further comprising:
determining that the medical patient is a member of a predefined insurance service;
determining a procedure associated with the identified gaps in care;
generate a pre-approval for the procedure based at least in part on the medical patient being the member of the predefined insurance service; and
wherein the notification indicates the pre-approval.

18. The system of claim 13, the operations further comprising:
determining an impact of a data type on the confidence value;
determining that the impact satisfies a threshold impact; and
weighting the data type based at least in part on the impact satisfying the threshold impact

19. The system of claim 13, the operations further comprising:
receiving feedback data associated with the notification;
determining, utilizing the feedback data and the machine learning models, a data type that impacts identification of the gap in care; and
updating the machine learning models to determine the gap in care based at least in part on the data type.

20. The system of claim 13, the operations further comprising weighting the first data prior to training the machine learning models, the weighting based at least in part on at least one of an age of the medical patient, a gender of the medical patient, diagnoses associated with the medical patient, medication use data associated with the medical patient, or procedure history associated with the medical patient.
